(19) United States
(12) Patent Application Publication
(10) Pub. No.: US 2019/0350476 A1
(43) Pub. Date: Nov. 21, 2019

(54) HEART RATE MONITOR ELECTRODE AND METHOD FOR MEASURING HEART RATE

(71) Applicant: ARIONEO, Mont Pres Chambord (FR)

(72) Inventors: Thomas Buisson, Clamart (FR); Guillaume DUBOIS, Paris (FR); Erwan MELLERIO, Mont Pres Chambord (FR); Soumaya YAZIDI, Puteaux (FR)

(21) Appl. No.: 16/347,890

(22) PCT Filed: Nov. 6, 2017
(86) PCT No.: PCT/FR2017/053028

§ 371 Date: May 7, 2019

(30) Foreign Application Priority Data
Nov. 9, 2016 (FR) 1660836

(51) Int. Cl. A61B 5/0408

(52) U.S. Cl. CPC A61B 5/0408; A61B 5/02405

(57) ABSTRACT

The invention relates to a heart rate monitor electrode (1). The electrode comprises a mounting (2), conductive points (23) and a plate of absorbent material (4). The free end (24) of at least some of the conductive points (23) is flush with the contact surface of the plate of absorbent material (4) or stands proud from same when the plate of absorbent material (4) is in at least partial compressed form and releases at least a portion of the liquid it contains. According to other aspects, the invention relates to a device comprising at least one such electrode (1) and a method for measuring heart rate by means of the same.

The invention relates to the field of heart rate measurement. Notably, the invention relates to an electrode and a method for measuring the heart rate of animals with fur.

A device for measuring heart rate comprising a basin with a flexible side wall into which is placed a sponge soaked in a gel or any other malleable material that is a good conductor of electricity is known, for example from document FR 2531330. This sponge then enables an electrical signal to be conducted between the animal and a chip connected to an electricity conducting cable.

The measurement and processing of a signal representing heart rate does not always give a reliable result. The quality of the result from this type of measurement and processing depends a lot on the quality of the transmission of the electrical signal propagating on the surface of the skin of the animal between said skin and the electrode designed to capture this signal. One objective of the invention is to improve on the electrodes of the prior art in order to obtain a better signal-to-noise ratio.

For this purpose, the invention proposes a heart rate monitor electrode comprising:
electrically conductive points, each extending from a free end to an electrically conductive circuit electrically linking the points to one another,
a soft plate of absorbent material that has a contact surface and can change shape elastically between
   an at least partially expanded form in which the plate of absorbent material is able to retain a liquid and
   an at least partially compressed form in which the plate of absorbent material occupies a lesser volume than in the expanded form and in which at least a portion of the liquid contained in the plate of absorbent material in the at least partially expanded form is released.

The free end of at least some of the points is flush with the contact surface of the plate of absorbent material or projects beyond same when the plate of absorbent material is in an at least partially compressed form, and releases at least a portion of the liquid that it contains when in its at least partially expanded form.

In this document, a circuit is not necessarily a set of conductive strips. A conductive flange or a flange with a conductive face also forms a circuit in this sense that it electrically connects the points together.

Such arrangements ensure that the free end of at least some of the points can come into direct contact with the skin of the animal, passing through the animal's fur, to ensure optimized conduction of the signal captured on the surface of its skin. The fact that the absorbent material can be deformed elastically is also advantageous. Indeed, the electrode is not a single-use electrode and can be reused several times. The absorbent material can be refilled with liquid before each new use without necessarily having to change the absorbent material. Furthermore, compressing the absorbent material releases the liquid, which can further improve the direct contact between the skin and the free end of the points passing through the animal's coat. Thus, the electrical conduction between the skin and the points can be increased, even if the animal is not moving and has not yet started to sweat. The signal-to-noise ratio of the electrical signals captured by the electrode is therefore improved and optimized.

The electrode according to the invention also has one or other of the following features taken individually or in combination with one or more other features:
the thickness of the plate of absorbent material varies when the plate of absorbent material changes from the at least partially expanded form to the at least partially compressed form thereof, the points extending in a compression direction essentially parallel this thickness, the electrode having an elastic mechanism making it possible to move the points in relation to the plate of absorbent material parallel to the compression direction,
the electrode has a flattened casing with two main faces,
   a first main face with an opening designed to face an animal skin where the heart rate is to be captured, this opening enabling the passage of the free end of the points, and
   a second main face with a pressure surface cooperating with the elastic mechanism to compress the plate of absorbent material in the compression direction when a pressure is exerted on the second main face of the electrode,
the electrode includes peripheral points of which the free ends are essentially arranged in a plane perpendicular to the compression direction and in a circle in this plane, with none of the free ends of the points being arranged outside this circle,
each point is surrounded by a portion of the plate of absorbent material,
at least the free end of the points is coated with silver or a silver alloy,
the points, and possibly the electrically conductive circuit to which the points are electrically connected, are made of a conductive elastic material, for example a polymer doped with electrically conductive particles.

According to another aspect, the invention relates to a heart rate monitor device including a receiver designed to receive the signal captured by at least one electrode having one or other of the aforementioned features, taken individually or in combination with one or more other features.

Advantageously, a transmitter electrically linked to at least one electrode and designed to communicate with the receiver is then placed in a casing raised above a flange rigidly connected to an electrode, a space thus being formed between the casing and the flange for the passage of a strap designed to hold the electrode and the casing on an animal, the electrode being placed in contact with the skin of the animal.

According to yet another aspect, the invention relates to a method for measuring the heart rate of an animal comprising the following steps:
providing an electrode as claimed in one of the preceding claims,
at least partially soaking the plate of absorbent material of this electrode with a liquid, and
positioning and holding this electrode against the skin of the animal using holding and tightening means.

This method can include a step for adjusting the holding and tightening means on the animal, in which step the plate of absorbent material is compressed and releases at least a portion of the liquid contained therein.

Other features and advantages of the invention will become apparent on reading the detailed description below and the attached drawings. In these drawings:

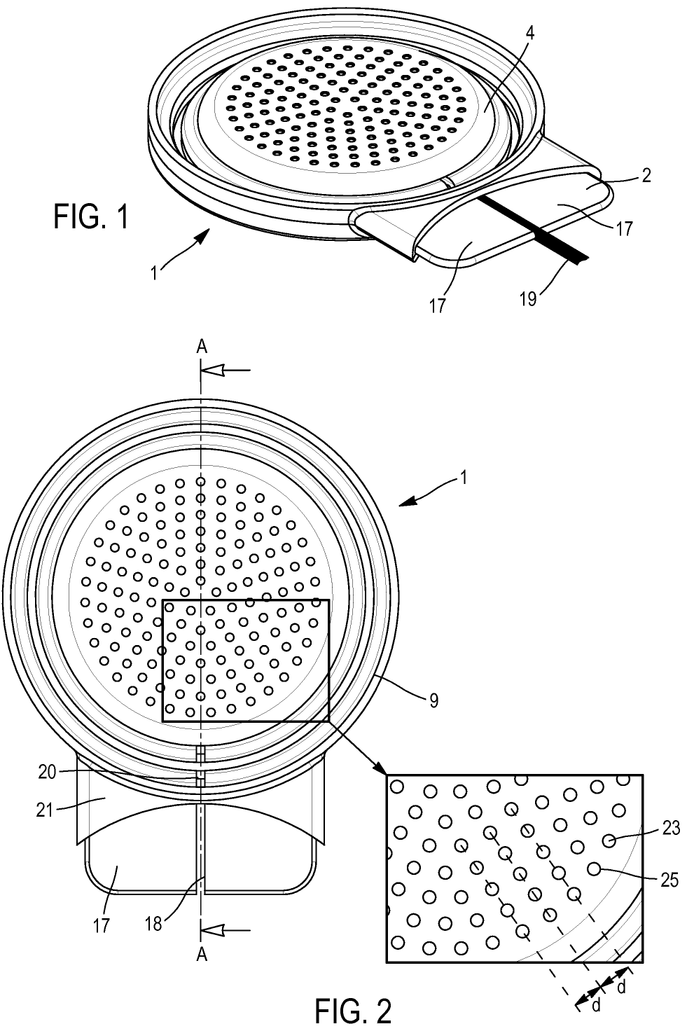

FIG. 1 is a schematic perspective view of an example embodiment of a heart rate monitor electrode according to the invention,

FIG. 2 is a schematic top view of the example embodiment of the heart rate monitor electrode shown in FIG. 1, with an insert showing a magnification of a portion of this figure,

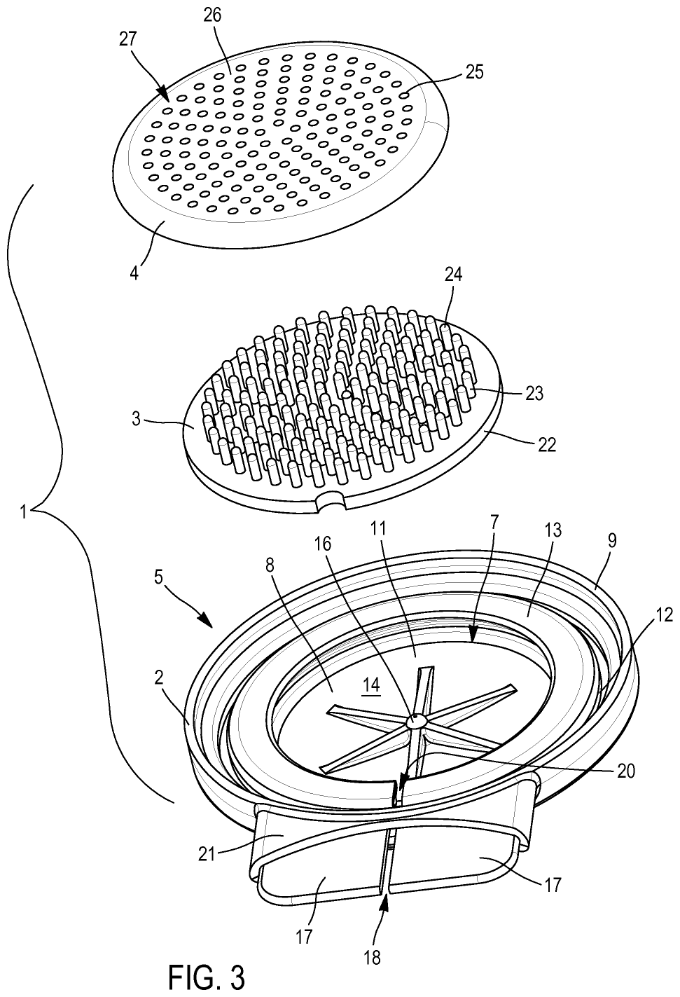

FIG. 3 is an exploded schematic perspective view of different elements of the electrode in FIGS. 1 and 2,

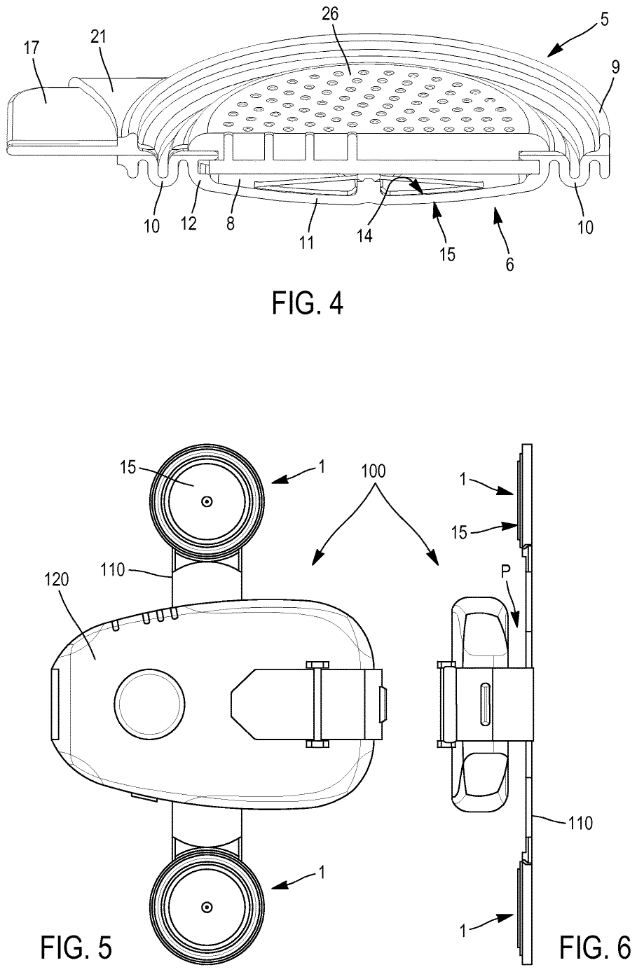

FIG. 4 is a schematic cross-sectional view taken along the line A-A in FIG. 2 of the embodiment of the heart rate monitor electrode shown in FIGS. 1 to 3, and

FIGS. 5 and 6 are schematic front and side views respectively of a heart rate monitor device with two electrodes such as those shown in FIGS. 1 to 4.

The same reference signs are used to indicate identical or similar elements in the figures.

An example embodiment of an electrode is described below with reference to FIGS. 1 to 4.

This electrode 1 comprises a support 2, a conductive plate 3 and a plate of absorbent material 4.

The support 2 is for example made of plastic. The support has a flattened shape with a first main face 5 and a second main face 6 (see FIG. 4). The first main face 5 has an opening 7 designed to face an animal skin where the heart rate is to be captured.

The support 2 has a basin 8 linked elastically to an external annular frame 9 by a soft elastic membrane 10. The elastic membrane 10 therefore forms an elastic mechanism or elastic means enabling the basin 8 to move in relation to the frame 9. The basin 8 is essentially round. The basin has a bottom 11 and a wall 12 extending between the bottom 11 and an upper edge 13 delimiting the opening 7. The bottom 11 is concave. The bottom has an inner surface 14 and an outer surface 15. The outer surface 15 is a pressure surface. The inner surface 14 has a protuberance 16. This protuberance 16, in the example shown here, is in the center of a six-pointed star strengthening and stiffening the bottom 11 of the basin 8.

The edge of the frame 9 in contact with the skin is raised in relation to the upper edge 13 of the basin 8 by a distance of for example between 1 mm and 3 mm, and more specifically 2 mm.

Two tabs 17 extend radially towards the outside of the support 2 from the frame 9. A channel 18 is formed between the two tabs 17 for the passage of a cable 19 (see FIG. 1), for example a shielded cable, or another conductive element. This channel 18, which is extended by a slot 20 in the frame 9 and in the wall 12 and the upper edge 13 of the basin 8, also allows separation between the portions of the upper edge 13 of the basin 8 located on either side of the slot 10, to facilitate the installation of the conductive plate 3 in the basin 8 by clipping. To maintain the separation between the tabs 17, a band 21, which may be removable, extends around the tabs 17 and above and below a portion of the channel 18.

The conductive plate 3 has an electrically conductive circuit in the form of a flange 22 that is essentially round and flat. Electrically conductive points 23 each extend from a free end 24 to the flange 22. This flange 22 may be a one-piece part made of a conductive metal, or a printed circuit, or any other means enabling the conductive points to be electrically connected to one another in an electrically continuous circuit. Thus, alternatively, the points 23 and/or the electric circuit 22 to which the points are electrically connected are made of a polymer doped with electrically conductive particles. This polymer is for example polydimethylsiloxane mixed with carbon powder (45% of the total volume) with a grain size of 40 μm or 40 nm carbon nanotubes (3% of the total volume). Nanometric or micrometric metal fibers can also be added to the polymer, such as silver nanofibers with a diameter of 30 nm, for example.

Each conductive point 23 is for example a cylinder with a rounded surface at the free end 24 thereof. Each conductive point 23 is for example a conductive metal or any other material enabling electrical conduction between the free end 24 thereof and the electrical circuit or flange 22 linking the conductive points 23 to one another (for example, a conductive point 23 can be made of a plastic coated with one or more conductive materials, or a conductive metal such as copper with the free end 24 coated with another conductive metal such as silver or a silver alloy, a polymer doped with conductive particles as mentioned above, etc.). In one embodiment, the conductive points 23 are made of sections of silver wires welded onto a printed-circuit (copper sheet on insulator) flange 22. In another embodiment, the flange 22 and the conductive points 23 form a cast single part, for example made of silver (or a silver alloy) or aluminum (or an aluminum alloy) coated with silver (or a silver alloy). In yet another embodiment, the flange 22 and the conductive points 23 form a single molded part formed from a polymer doped with conductive particles as mentioned above.

The height of the conductive points 23 above the flange 22 is for example between 2 mm and 4 mm, and more specifically 3 mm. All of the free ends 24 of the conductive points 23 are in the same plane. Specifically, said points are designed to come into contact with the skin of an animal and should be felt by the animal as little as possible, so as to minimize or eliminate the discomfort caused to the animal. For this reason also, the conductive points 23 are arranged relatively uniformly inside a circle. Preferably, the conductive points 23 are distributed in concentric circles. The circles are spaced apart from one another by a distance of between 2 mm and 3 mm, and more specifically 2 mm. The distance d between two points in a circle is between 2 mm and 3 mm, and preferably 2 mm. In other words, the density of the points is for example between one point per 10 mm² and one point per 40 mm², and more preferably equal or close to one point per 16 mm². The circle in which all of the peripheral (outermost) points are located has for example a radius of between 20 mm and 40 mm, and more specifically 30 mm. The peripheral points, located on this circle, are thus arranged in a uniform manner. This uniform circular arrangement with no projecting edges prevents or limits any sensations of discomfort that may be caused to the animal by the conductive points 23.

The electrical circuit of the flange 22 is electrically connected to a transmitter (not shown) by the cable 19 or any other connection means.

The plate of absorbent material 4 is flexible and hard-wearing. The plate is for example made of polypropylene or polyurethane foam or any other material, for example a spongy material, designed to be soaked in a liquid and release said liquid when compressed. The plate of absorbent material 4 has a flattened round shape. The plate for example has a diameter of between 30 mm and 50 mm, more specifically 36 mm, and a thickness of between 3 mm and 6 mm, and more specifically equal to the height of the conductive points 23.

The plate of absorbent material 4 is pierced, through the thickness thereof, by holes 25 having a diameter and arrangement designed to enable the conductive points 23 to pass through. Each conductive point 23 is surrounded by a portion of the plate of absorbent material 4. Thus, the salient appearance of the conductive points 23 is attenuated by the plate of absorbent material 4 both for the animal and for the person handling the electrode 1. Preferably, when the plate of absorbent material 4 is not compressed, the free end 24 of the conductive points 23 does not project beyond the holes 25 (the free end 24 of the conductive points 23 can be just flush with the surface 26 of the outer face 27 of the plate of absorbent material 4). Conversely, this surface 26 is substantially at the same level as the edge of the frame 9 (see FIG. 4).

When the plate of absorbent material 4, the conductive plate 3 and the support 2 are assembled together, the conductive points 23 extend in a compression direction essentially parallel to the thickness of the plate of absorbent material 4.

The edge of the plate of absorbent material is rounded and can possibly be slid at least partially beneath the edge 13 of the basin 8 in order to hold the plate of absorbent material 4 on the support 2. Once the plate of absorbent material 4 is in place in the basin 8, said plate blocks the opening 7 of the basin 8.

A heart rate monitor device 100 is shown in FIGS. 5 and 6. The device in particular has a flange 110 on which two electrodes 1 and a casing 120 are mounted. The flange 110 is for example made of a flexible plastic and has electrically conductive elements enabling the electrodes 1 to be connected to an electronic circuit (not shown) arranged in the casing 120. The electrodes 1 and the casing 120 can be respectively connected to these electrically conductive elements using cables, which may be shielded, or any other suitable connection (for example mini coaxial connectors for high-frequency signals, such as the U-FL model marketed by Hirose). Advantageously, the electrodes 1 and the casing 120 can be assembled removably on the flange 110 to enable either one of these elements to be swapped individually if damaged or worn out, or to temporarily remove the casing 120 without removing the entire device 100 from the animal, etc.

The casing 110 is for example made of a plastic and is sufficiently fluid-tight to be able to be wetted without damaging the electrical circuit contained therein. The electronic circuit housed therein includes a transmitter designed to communicate data captured by different sensors, and in particular by the electrodes 1, with a receiver over a wireless link.

The casing 120 is raised above the flange 110 in order to leave a space P between the casing 120 and the flange 110 for the passage of the holding and tightening means. These holding and tightening means have for example a strap (not shown) enabling the device 100 to be positioned and held on the animal. This space or passage P extends transversely in relation to the flange 110. Thus, the strap passes beneath the casing 120 and covers the electrodes 1 and presses only same into contact with the skin of the animal.

Before the device 100 is placed on the animal, the device 100, or at least the plates of absorbent material 4, are wetted for example with water in order to impregnate and soak the plate of absorbent material 4 with same. The plate of absorbent material 4 is then essentially in the expanded form and retains water. When tightening the strap, which is in contact with the outer surface 15 of the basin 8, the strap exerts pressure on the bottom 11 of the basin. The basin 8 as a whole is moved towards the skin of the animal since the edge of the frame 9, which is in contact with the skin, is raised above the edge 13 of the basin 8. Furthermore, since the outer surface 26 of the plate of absorbent material 4 is substantially at the same level as the edge of the frame 9, the plate of absorbent material 4 is compressed between the flange 22 compressed by the protuberance 16 and the skin. The absorbent material releases at least a portion of the water previously absorbed. The conductive points 23 then come out of the holes 25 in the plate of absorbent material 4 and pass through the fur of the animal, then at least some of said points come into contact with the epidermis of the animal. Electrical conduction can then be established between the epidermis and the electronic circuit.

1. A heart rate monitor electrode comprising: electrically conductive points, each extending from a free end to an electrically conductive circuit electrically linking the points to one another, a soft plate of absorbent material that has a contact surface and can change shape elastically between an at least partially expanded form in which the plate of absorbent material is able to retain a liquid and an at least partially compressed form in which the plate of absorbent material occupies a lesser volume than in the expanded form and in which at least a portion of the liquid contained in the plate of absorbent material in the at least partially expanded form is released, characterized in that the free end of at least some of the points is flush with the contact surface of the plate of absorbent material or projects beyond same when the plate of absorbent material is in an at least partially compressed form, and releases at least a portion of the liquid that it contains when in its at least partially expanded form.

2. The electrode as claimed in claim 1, in which the thickness of the plate of absorbent material varies when the plate of absorbent material changes from the at least partially expanded form to the at least partially compressed form thereof, the points extending in a compression direction essentially parallel this thickness, the electrode having an elastic mechanism making it possible to move the points in relation to the plate of absorbent material parallel to the compression direction.

3. The electrode as claimed in claim 2, including a flattened support with two main faces, a first main face with an opening designed to face an animal skin where the heart rate is to be captured, this opening enabling the passage of the free end of the points, and a second main face with a pressure surface cooperating with the elastic mechanism to compress the plate of absorbent material in the compression direction when a pressure is exerted on the second main face of the electrode.

4. The electrode as claimed in one of claim 2, including peripheral points of which the free ends are essentially arranged in a plane perpendicular to the compression direction and in a circle in this plane, with none of the free ends of the points being arranged outside this circle.

5. The electrode as claimed in claim 1, in which each point is surrounded by a portion of the plate of absorbent material.

6. The electrode as claimed in claim 1, in which at least the free end of the points is coated with silver or a silver alloy.

7. The electrode as claimed in claim 1, in which the points and the electrically conductive circuit to which the points are electrically connected are made of a polymer doped with electrically conductive particles.

8. A heart rate monitor device having at least one electrode as claimed in claim 1 and a receiver designed to receive the signal captured by this electrode.

9. The heart rate monitor device as claimed in claim 8, in which a transmitter electrically linked to at least one electrode and designed to communicate with the receiver is placed in a casing raised above a flange rigidly connected to an electrode, a space (P) thus being formed between the casing and the flange for the passage of a strap designed to hold the electrode and the casing on an animal, the electrode being placed in contact with the skin of the animal, wherein the electrode comprises a heart rate monitor electrode that comprises electrically conductive points, each extending from a free end to an electrically conductive circuit electrically linking the points to one another, a soft plate of absorbent material that has a contact surface and can change shape elastically between an at least partially expanded form in which the plate of absorbent material is able to retain a liquid and an at least partially compressed form in which the plate of absorbent material occupies a lesser volume than in the expanded form and in which at least a portion of the liquid contained in the plate of absorbent material in the at least partially expanded form is released, characterized in that the free end of at least some of the points is flush with the contact surface of the plate of absorbent material or projects beyond same when the plate of absorbent material is in an at least partially compressed form, and releases at least a portion of the liquid that it contains when in its at least partially expanded form.

10. A method for measuring the heart rate of an animal comprising: providing an electrode as claimed in claim 1, at least partially soaking the plate of absorbent material of this electrode with a liquid, and positioning and holding this electrode against the skin of the animal using holding and tightening means.

11. The method for measuring the heart rate as claimed in claim 10, including a step for adjusting the holding and tightening means on the animal, in which step the plate of absorbent material is compressed and releases at least a portion of the liquid contained therein.
